SEQUENCE LISTING (52 sequences)

SEQ ID NO: 1 - protein, 29 aa, Hepatitis C virus (20-48)
QDVKFPGGGQIVGGVYLLPRRGPRLGVRA

SEQ ID NO: 2 - protein, 27 aa, Hepatitis C virus (27-53)
GGQIVGGVYLLPRRGPRLGVRATRKTS

SEQ ID NO: 3 - protein, 26 aa, Hepatitis C virus (35-60)
YLLPRRGPRLGVRATRKTSERSQPRG

SEQ ID NO: 4 - protein, 19 aa, Hepatitis C virus (68-86)
ARRPEGRTWAQPGYPWPLT

SEQ ID NO: 5 - protein, 21 aa, Hepatitis C virus (84-104)
PLYGNEGCGWAGWLLSPRGSR

SEQ ID NO: 6 - protein, 23 aa, Hepatitis C virus (114-136)
RRSRNLGKVIDTLTCGFADLMGY

SEQ ID NO: 7 - protein, 22 aa, Hepatitis C virus (128-149)
AGFADLMGYIPLVGAPLGGAAR

SEQ ID NO: 8 - protein, 19 aa, Hepatitis C virus (154-172)
GVRVLEDGVNYATGNLPGA

SEQ ID NO: 9 - protein, 26 aa, Hepatitis C virus (1008-1033)
REILLGPADGMVSKGWRLLAPITAYA

SEQ ID NO: 10 - protein, 27 aa, Hepatitis C virus (1016-1042)
DGMVSKGWRLLAPITAYAQQTRGLLGA

SEQ ID NO: 11 - protein, 27 aa, Hepatitis C virus (1077-1103)
VCWTVYHGAGTRTIASPKGPVIQMYTN

SEQ ID NO: 12 - protein, 24 aa, Hepatitis C virus (1088-1111)
RTIASPKGPVIQMYTNVDDQLVGW

SEQ ID NO: 13 - protein, 21 aa, Hepatitis C virus (1150-1170)
GSLLSPRPISYLKGSSGGLPL

SEQ ID NO: 14 - protein, 26 aa, Hepatitis C virus (1191-1216)
KAVDFIPVENLETTMRSPVFTDNSSP

SEQ ID NO: 15 - protein, 25 aa, Hepatitis C virus (1205-1229)
MRSPVFTDNSSPPVVPQSQFVAHLH

SEQ ID NO: 16 - protein, 27 aa, Hepatitis C virus (1242-1268)
AAYAAQGYKVLVLNPSVATALGFGAYM

SEQ ID NO: 17 - protein, 22 aa, Hepatitis C virus (1283-1304)
RTITTGSPITYSTYGKFLDAGG

SEQ ID NO: 18 - protein, 31 aa, Hepatitis C virus (1324-1354)
TSILGIGTVLDQAETAGARLVVLATATPPGS

SEQ ID NO: 19 - protein, 28 aa, Hepatitis C virus (1405-1432)
AKLVALGINAVAYYRGLDVSVIPTSGDV

SEQ ID NO: 20 - protein, 27 aa, Hepatitis C virus (1423-1450)
VSVIPTSGDVVVATDALMTGYTGDFDS

SEQ ID NO: 21 - protein, 33 aa, Hepatitis C virus (1563-1595)
TGLTHIDAHFLSQTKQSGENLPYLVAYQATVAA

SEQ ID NO: 22 - protein, 28 aa, Hepatitis C virus (1712-1739)
SQHLPYIEQGMMLAEQFKKQALGLLQTA

SEQ ID NO: 23 - protein, 35 aa, Hepatitis C virus (1744-1778)
EVIAPAVQTNWQKLETFWAKHMWNFISGIQYLAGL

SEQ ID NO: 24 - protein, 34 aa, Hepatitis C virus (1785-1818)
PAIASLMAFTAAVTSPLTTSQTLLFNILGGWVAA

SEQ ID NO: 25 - protein, 28 aa, Hepatitis C virus (1813-1841)
GWVAAQLAAPGAATAFVGAGLAGAAIGS

SEQ ID NO: 26 - protein, 36 aa, Hepatitis C virus (1827-1862)
TAFVGAGLAGAAIGSVGLKGVLIDILAGYGAGVAGA

SEQ ID NO: 27 - protein, 29 aa, Artificial sequence(20-48)
  Variant 4,(6).(10),13,14,(19).(21),23,24,26: Xaa = any amino acid
QDVXFXXXXXIVXXVYLLXXXGXXLXVRA

SEQ ID NO: 28 - protein, 27 aa, Artificial sequence (27-53)
  Variant 6,7,(12).(14),16,17,(19).(22),(24).(26): Xaa = any amino acid
GGQIVXXVYLLXXXGXXLXXXXTXXXS

SEQ ID NO: 29 - protein, 26 aa, Artificial sequence (35-60)
  Variant (4).(6),9,11,(13).(20): Xaa = any amino acid
YLLXXXGPXLXVXXXXXXXXRSQPRG

SEQ ID NO: 30 - protein, 19 aa, Artificial sequence (68-86)
  Variant (10).(15),17: Xaa = any amino acid
ARRPEGRTWXXXXXXWXLT

SEQ ID NO: 31 - protein, 21 aa, Artificial sequence (84-104)
  Variant (4).(6),8,9,12,(15).(21): Xaa = any amino acid
PLYXXXGXXWAXWLXXXXXXX

SEQ ID NO: 32 - protein, 23 aa, Artificial sequence (114-136)
  Variant 7,11,12,(14).(16),19,20: Xaa = any amino acid
RRSRNLXKVIXXLXXXFAXXMGY

SEQ ID NO: 33 - protein, 22 aa, Artificial sequence (128-149)
  Variant 4,5,11,14,16,18,19: Xaa = any amino acid
AGFXXLMGYIXLVXAXLXXAAR

SEQ ID NO: 34 - protein, 19 aa, Artificial sequence (154-172)
  Variant 3,(6).(8),10,(13).(15),17: Xaa = any amino acid
GVXVLXXXVXYAXXXLXGA

SEQ ID NO: 35 - protein, 26 aa, Artificial sequence (1008-1033)
  Variant 6,7,9,(13).(15),17,21,23: Xaa = any amino acid
REILLXXAXGMVXXXWXLLAXIXAYA

SEQ ID NO: 36 - protein, 27 aa, Artificial sequence (1016-1042)
  Variant (4).(7),9,13,(19).(24): Xaa = any amino acid
DGMXXXXWXLLAXITAYAXXXXXXLGA

SEQ ID NO: 37 - protein, 27 aa, Artificial sequence (1077-1103)
  Variant 4,8,(11).(13),(15).(18),20,21: Xaa = any amino acid
VCWXVYHXAGXXXIXXXXGXXIQMYTN

SEQ ID NO: 38 - protein, 24 aa, Artificial sequence (1088-1111)
  Variant (4).(7),9,12,16,(18).(20): Xaa = any amino acid
RTIXXXXGXVIXMYTXVXXXLVGW

SEQ ID NO: 39 - protein, 21 aa, Artificial sequence (1150-1170)
  Variant (5).(7),10,(13).(15),(17).(19): Xaa = any amino acid
GSLLXXXPIXYLXXXSXXXLL

SEQ ID NO: 40 - protein, 26 aa, Artificial sequence (1191-1216)
  Variant 4,7,9,10,(12).(14),(16).(18),20,(21).(23): Xaa = any amino acid
KAVXFIXVXXLXXXMXXXVXXXXSSP

SEQ ID NO: 41 - protein, 25 aa, Artificial sequence (1205-1229)
  Variant (7).(9),12,13,16,17,(19).(21),23: Xaa = any amino acid
MRSPVFXXXSSXXVVXXSXXXAXLH

SEQ ID NO: 42 - protein, 27 aa, Artificial sequence (1242-1268)
  Variant (4).(7),9,(14).(16),20,23,24: Xaa = any amino acid
AAYXXXXYXVLVLXXXVAAXLGXXAYM

SEQ ID NO: 43 - protein, 22 aa, Artificial sequence (1283-1304)
  Variant (4).(7),10,(12).(16),18: Xaa = any amino acid
RTIXXXXPIXYXXXXXFXADGG

SEQ ID NO: 44 - protein, 31 aa, Artificial sequence (1324-1354)
  Variant 5,7,11,12,14,25,(28).(30): Xaa = any amino acid
TSILXIXTVLXXAXTAGARLVVLAXATXXXS

SEQ ID NO: 45 - protein, 28 aa, Artificial sequence (1405-1432)
  Variant 5,7,9,10,15,16,18,20,23,(25).(27): Xaa = any amino acid
AKLVXLXIXXVAYYXXLXVXVIXTXXXV

SEQ ID NO: 46 - protein, 27 aa, Artificial sequence (1423-1450)
  Variant (5).(7),14,15,19,20,23,24: Xaa = any amino acid
VSVIXXXGDVVVAXXALMXXYTXXFDS

SEQ ID NO: 47 - protein, 33 aa, Artificial sequence (1563-1595)
  Variant 4,7,9,(12).(19),21,25,27,28,30: Xaa = any amino acid
TGLXHIXAXFLXXXXXXXXNLXYLXAXXAXVAA

SEQ ID NO: 48 - protein, 28 aa, Artificial sequence (1712-1739)
  Variant 5,(8).(10),15,16,(18).(20),(22).(24): Xaa = any amino acid
SQHLXYIXXXMMLAXXFXXXAXXXLQTA

SEQ ID NO: 49 - protein, 35 aa, Artificial sequence (1744-1778)
  Variant 4,5,(8).(10),12,13,15,20,21,(28).(32),34: Xaa = any amino acid
EVIXXAVXXXWXXLXTFWAXXMWNFISXXXXXAXL

SEQ ID NO: 50 - protein, 34 aa, Artificial sequence (1785-1818)
  Variant 4,5,10,(14).(16),(19).(21),26,28: Xaa = any amino acid
PAIXXLMAFXAAVXXXLTXXXTLLFXIXGGWVAA

SEQ ID NO: 51 - protein, 28 aa, Artificial sequence (1813-1841)
  Variant 4,6,9,,10,13,14,18,20,21,23: Xaa = any amino acid
GWVXAXLAXXGAXXAFVXAXXAXAAIGS

SEQ ID NO: 52 - protein, 36 aa, Artificial sequence (1827-1862)
  Variant 5,7,10,14,15,17,19,20,24,(28).(30),32: Xaa = any amino acid
TAFVXAXLAXAAIXXVXLXXVLIXILAXXXAXVAGA

(19) United States
(12) Patent Application Publication
(10) Pub. No.: US 2003/0194747 A1
(43) Pub. Date: Oct. 16, 2003

(54) Convergent combinatory peptide libraries and their use for vaccination against hepatitis c virus

(76) Inventors: Bertrand Georges, Lille (FR); Helene Gras-Masse, Lille (FR); Ahmil Bouzidl, Lille (FR); Claude Auriault, Lille (FR)

Correspondence Address: BIRCH STEWART KOLASCH & BIRCH, PO BOX 747, FALLS CHURCH, VA, 22040-0747, US

(21) Appl. No.: 10/296,558

(22) PCT Filed: May 23, 2001
(86) PCT No.: PCT/FR01/01596

(30) Foreign Application Priority Data
May 26, 2000 (FR) 00/06744

(51) Int. Cl. G01N 33/53; C07H 21/04; C12P 21/02; C12N 5/06; C07K 14/74; C07K 14/005

(52) U.S. Cl. 435/7.1; 435/69.1; 435/456; 435/320.1; 435/325; 530/350; 536/23.5

(57) ABSTRACT

The invention relates to the design and synthesis of immunogenic peptides corresponding to restricted regions of virus proteins involving a T cell immune response and to convergent combinatory peptide libraries derived from said regions.

The invention applies, in particular, to the hepatitis C virus and to the production of corresponding vaccinal preparations.

The invention relates to the design and synthesis of mixtures of immunogenic peptides corresponding to restricted regions of virus proteins and, in particular, of the hepatitis C virus (HCV) and their use to produce vaccinal preparations against said viruses.

The hepatitis C virus was identified in 1989 (CHOO Q. L. et al., Science, 1989, 244, 359-361). It is the main causative agent of non-A, non-B chronic hepatitis parenterally transmitted.

Infection by the HCV virus develops into a chronic pathology in 60 to 80 percent of the cases. Such infections are highly prevalent in the population at large, being in the order of 1 to 2 percent in the industrialized countries, particularly in Western Europe (ALTER H. J. et al., Blood, 1995, 85,1681-1695).

At the present time, antiviral treatments (interferon, Ribavirine®) enable only 20% of the patients to be cured and, above all, no anti-HCV vaccine affords real protection from the disease.

The HCV virus is one of the viruses of the Flaviviridae family having an RNA genome encoding a single polyprotein which is cleaved into several sub-fragments: a capsid protein, two glycoproteins, E1 and E2, and 6 so-called non-structural proteins: NS2, NS3, NS4A, NS4B, NS5A, NS5B. These viral proteins are antigenic targets for the immune response: the E1 and E2 envelope proteins form targets for the neutralizing antibodies, and the non-structural proteins are capable of inducing a cytotoxic effector response. However, mutations occur within the HCV genome with high frequency and generate numerous quasi-species that make it easier for the virus to escape from the antiviral defenses of the host (WEINER A. J., Proc. Natl. Acad. Sci. USA, 1992, 92,2755).

The immune mechanisms responsible for eliminating the HCV virus have not yet been clearly established. Observation of the groups of patients, about 20%, who are capable of eliminating the virus spontaneously after a phase of acute infection has made it possible to highlight a strong correlation between progress towards recovery and considerable proliferation of the specific CD4 lymphocytes of viral antigens, in particular of the capsid-protein and of the NS3 and NS4 proteins (DIEPOLDER H. M., Lancet, 1995, 346:1006). A TH1 type response appears to be the basis for this immunity, probably by regulating the cytotoxic response mediated by the CD8 cells or by directly secreting different antiviral factors. This response must, nonetheless persist to permit longer-term control of the virus (GERLACH J. T. et al., Gastroenterology, 1999, 117:933).

Synthetic peptides obtained from the capsid proteins and NS3 and NS4 proteins have shown their ability to induce a T helper type response (DIEPOLDER, J. Virol., 1997, 71:6011). Inducing and maintaining the CD4 response through the use of peptidic epitopes obtained from these proteins of interest represent a promising strategy for inducing protective anti-HCV immunity. However, peptides are generally only slightly immunogenic and, above all, they are rarely capable of inducing an immune response independently of the restriction linked to the polymorphous molecules of the Major Histocompatibility Complex (MHC).

That is why the Applicants have set themselves the task of selecting peptides of interest, having T epitope properties, similar to those already described, and of increasing their immunogenic power by modifying their native sequence, at defined positions, with the dual aim of increasing their anchorage to the molecules of the MHC and of broadening their recognition by the receptors of the T cells.

Thus, for each peptide of interest, a combinatory library of peptides is created in which the native amino acid and the replacement amino acid chosen to create artificial degeneracy of the selected peptide coexist, at each of the chosen positions (which will be defined further on).

The mixtures of peptides thus created possess the same antigenic specificity as the native peptide. These mixtures of peptides are thus to be considered as convergent and will be referred to hereinafter as “convertopes”.

The present invention thus relates to the process for preparing said mixtures of immunogenic peptides.

The first step in the process concerns the strategy for selecting sequences potentially capable of inducing the activation of the CD4 cells. The selection process concerns regions of the capsid protein and the NS3 and NS4 proteins of the HCV virus; these regions are chosen for their high density of anchoring motifs for human class II molecules of the MHC.

The role of class II molecules of the MHC is to present the degraded antigens in the form of peptides to the CD4 T cells. Presentation of these degraded antigens or T epitopes to the T lymphocytes by the class II molecules of the MHC is a requisite condition for inducing an immune response.

The molecules of the MHC are polymorphic proteins, encoded by numerous alleles each of which has specific characteristics of interaction with the T epitopes. Crystallography studies of several human class II molecules of the MHC have made it possible, in particular, to highlight the modalities of specific interaction with the T epitopes.

There are several important criteria: on one hand, the length of the peptides must be greater than II residues to permit efficient interaction; on the other hand, certain residues of the antigenic peptide play a specific role in the interaction by interacting with micro-environments or anchoring pockets of the molecule of the MHC.

There are four anchoring pockets, known as P1, P4, P6, P9, that play a dominant role in the recognition, whatever the MHC allele. These anchoring pockets are independent of one another and possess special physico-chemical properties that permit the specific anchoring of side chains of the peptide.

The polymorphic residues of the molecules of the MHC are chiefly grouped together in the area of the anchoring pockets and thus influence the particular interaction characteristics of the peptidic antigens.

The characteristics of the anchoring pockets of these major alleles were identified by STURNIOLO (STURNIOLO T., Nature Biotech.; 1999, 17:555).

The Applicants have deduced therefrom criteria common to the majority of the class II MHC alleles that are applicable to the predictive search for anchoring pockets, within native protein sequences.

Thus, in a motif of 9 adjacent amino acids, the amino acid in position P1 is necessarily hydrophobic and is an aliphatic or aromatic amino acid, i.e. V, I, L, F, M, W or Y; the amino acids in positions P4, P6 and P9 are, preferably, I, V, L, M, F or A; A, P, G, S or T; and A, I, V, Y, L, F or M, respectively.

These criteria were applied, in a predictive, non-experimental manner, to identifying sequences of interest in the capsid, and NS3 and NS4 proteins, the sequences of which are available from the SWISS PROT data bank (the reference of which is annexed to the listing of the sequences).

Several potential anchoring motifs were identified that differ by only 1 or 2 amino acids from the criteria defined above.

The portions of protein sequences having a high density of these anchoring motifs were identified and selected. The peptide sequences chosen have a length of between 19 and 36 residues. They include at least 1 or 2 residues upstream of the first anchoring unit and downstream of the last anchoring unit. These ends can be extended by 1 to 6 additional natural amino acids in order to include into the sequence charged amino acids, capable of facilitating the solubility of sequences that are too rich in hydrophobic residues.

In all, a group of 26 sequences was chosen in this way. They are presented in the Annex under the heading “listing of sequences”. Sequences ID Nos. 1 to 8 are regions of the capsid protein, sequences ID Nos. 9 to 21 are regions of the NS3 protein and sequences ID Nos. 22 to 26 are regions of the NS4 protein.

The sequences of interest having been identified, the second step in the process according to the invention is to design, on the basis of these sequences, convergent combinatory peptide libraries (termed “convertopes”).

The choice of the amino acid substitutions aimed at creating the combinatory library is based on the molecular characteristics of recognition of the antigens by the immune receptors, in particular by the molecules of the Major Histocompatibility Complex (MHC) and by the T lymphocyte receptors and, more particularly, on the notion of degeneracy of these mechanisms of recognition by the immune system (see, in particular, Hemmer et al. Immunol. Today 1998, 19,163-168 “Probing degeneracy in T-cell recognition using peptide combinatorial libraries”).

The degeneracy sought after, according to the present invention, is non-natural degeneracy that differs from so-called natural degeneracy which is produced on the basis of existing mutations, present in the antigen sequences of different natural variants of the viruses (GRAS-MASSE H., Pept. Res., 1992, 5:211-216). This concept of non-natural degeneracy for the development of convergent peptide mixtures was initially suggested by GRAS-MASSE H. (GRAS-MASSE H., Curr. Opin. in Immunol., 1999, 11:223-228).

The aim of using said mixtures is, in particular, to stimulate a broader immune response, which will permit subsequent recognition of peptides that are related to, but different from, the native peptide and which could be encountered in natural variants of the virus.

In the process according to the invention, a first type of degeneracy is created for the purpose of increasing the ability of the epitopes to anchor to the molecules of the MHC.

In the anchoring motifs identified, one checks that the P4, P6 and P9 residues of the native sequence satisfy the common criteria defined earlier according to the study by Sturniolo. If the amino acid fails to satisfy these criteria, its substitution by a more compliant amino acid, in particular by an alanine, is contemplated.

A second type of degeneracy can be created for the purpose of increasing recognition of the epitopes by the receptors of the T cells; it concerns only those positions that play no part in anchoring to the molecules of the MHC, i.e. the residues other than 1, 4, 6 and 9. The substitutions contemplated here are based on a matrix of replaceability devised by Geysen (J. Mol. Recog. 1988, 1, 32) which takes into account the considerable degeneracy of recognition of the antigens by the T receptors. All the positions can give rise to substitution, except for residues common to 2 or more neighboring anchoring units the substitution of which would be incompatible with the neighboring unit or with a substitution already effected therein.

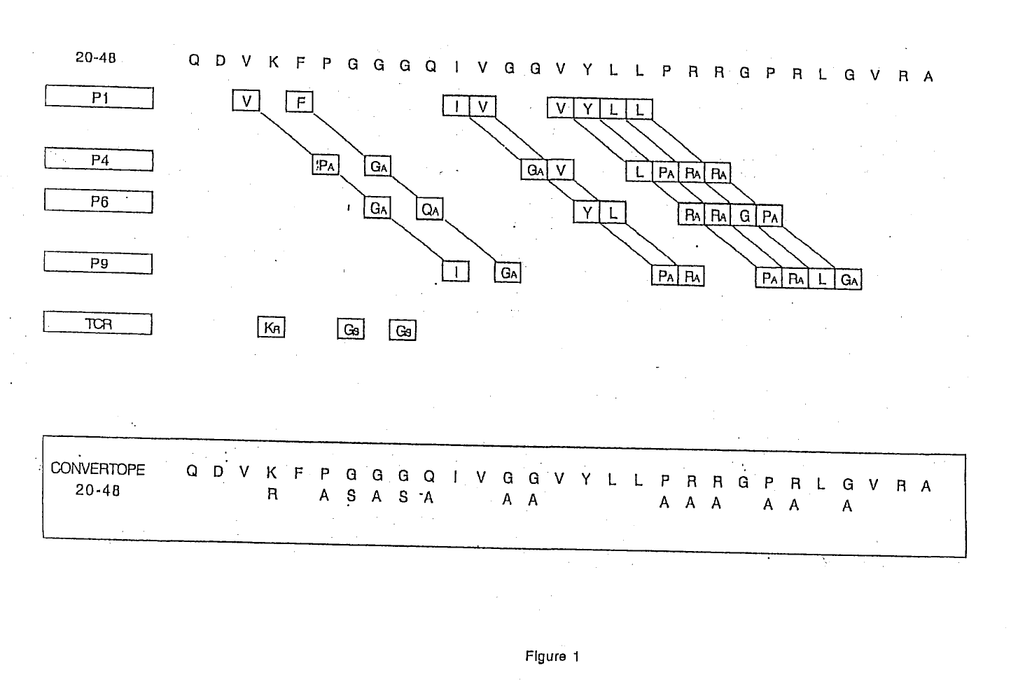

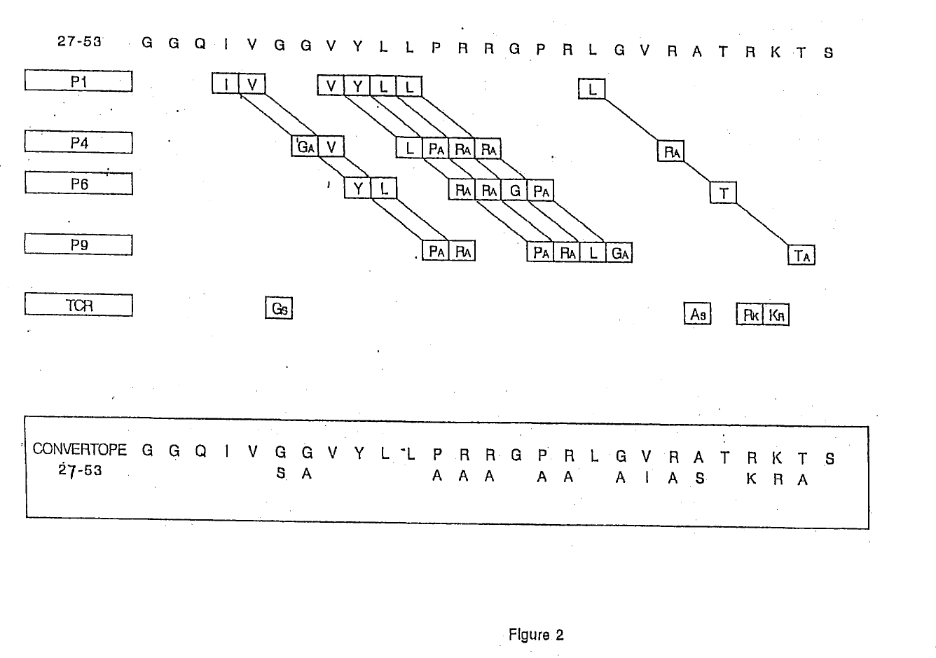

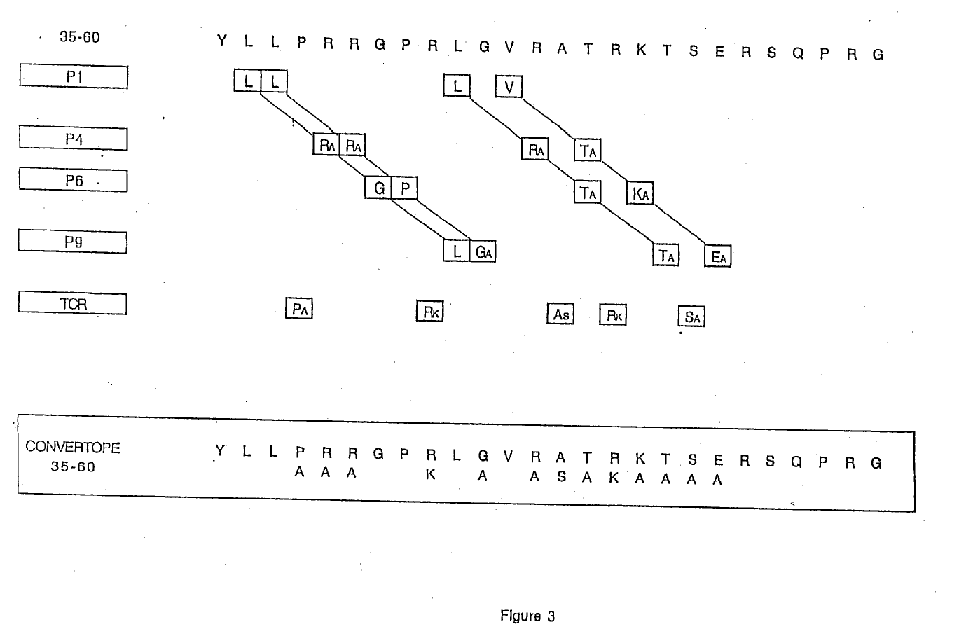

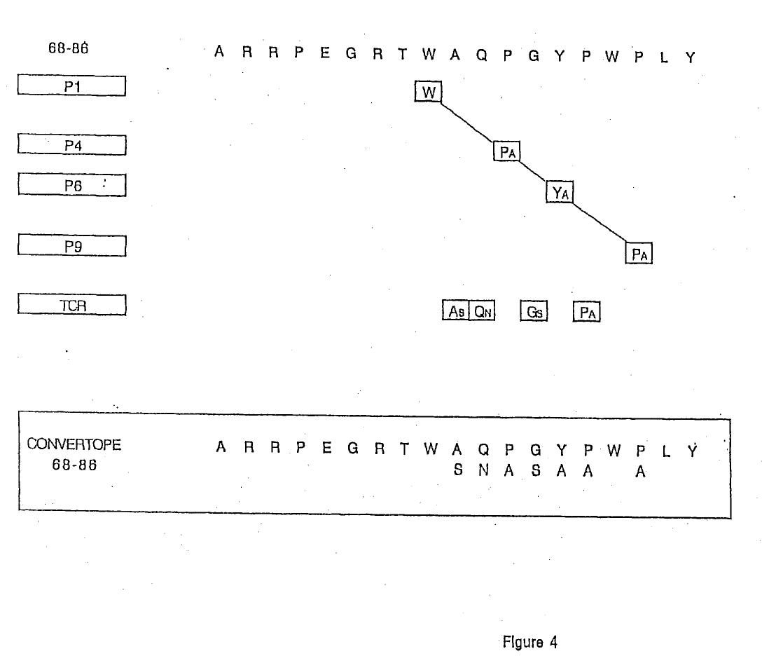

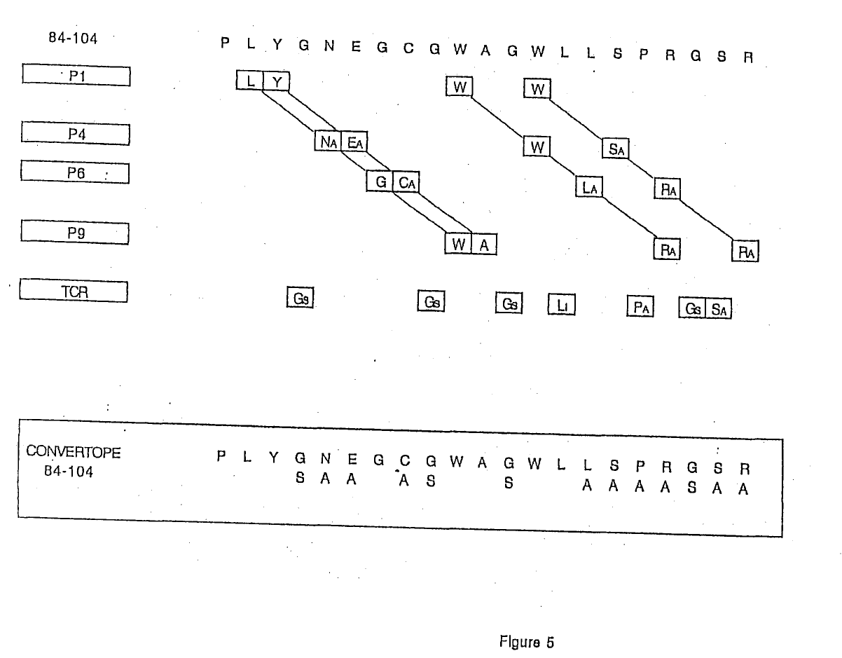

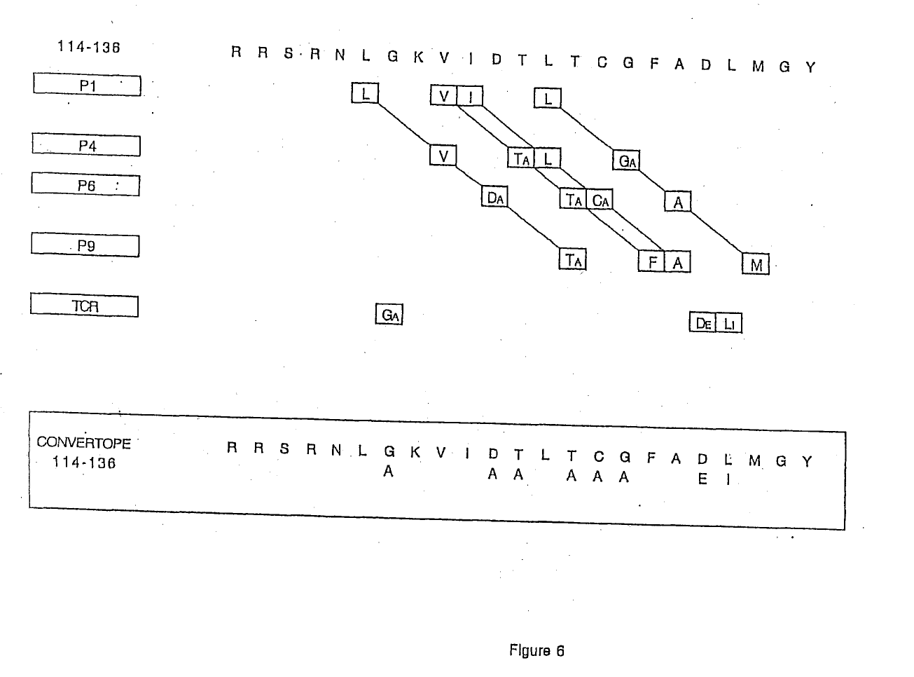

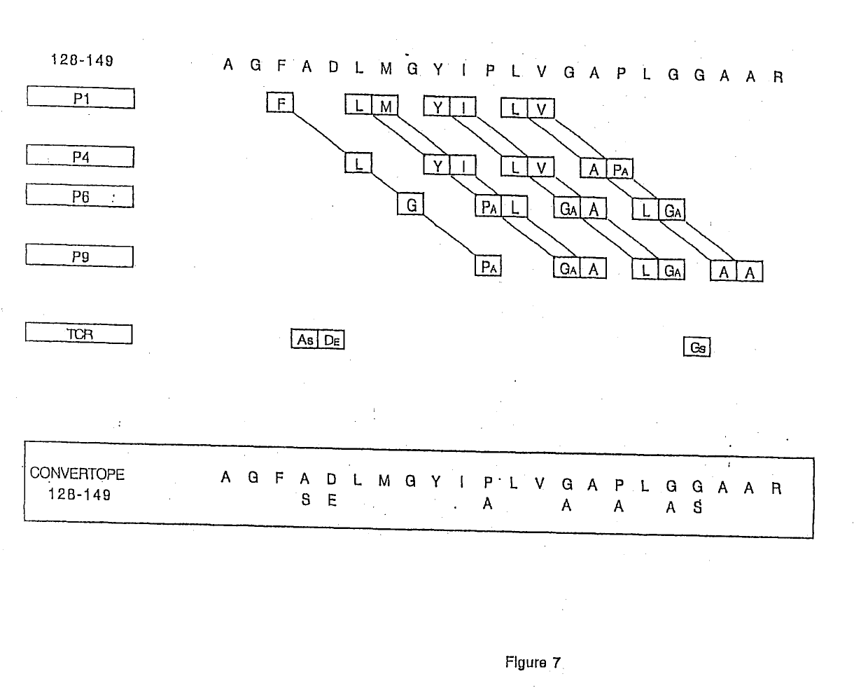

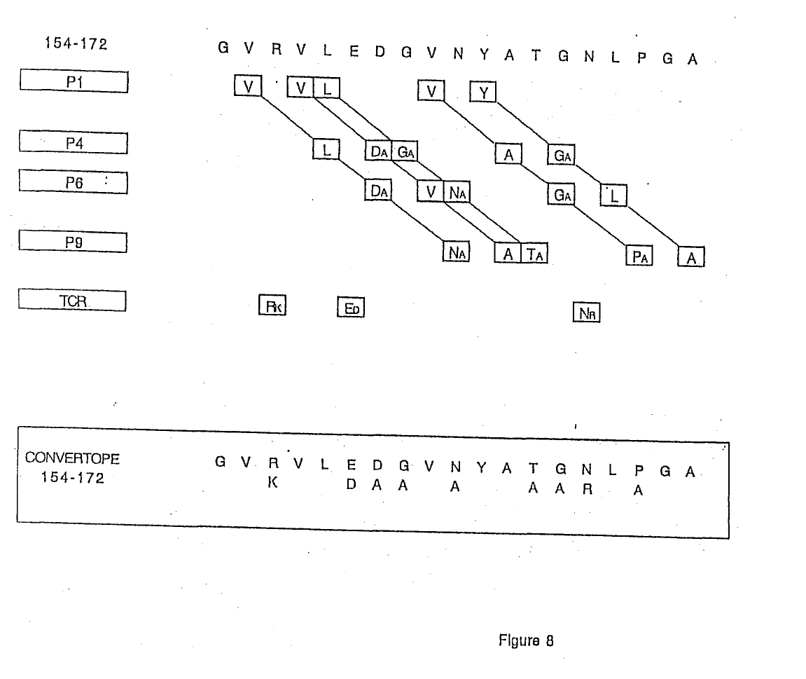

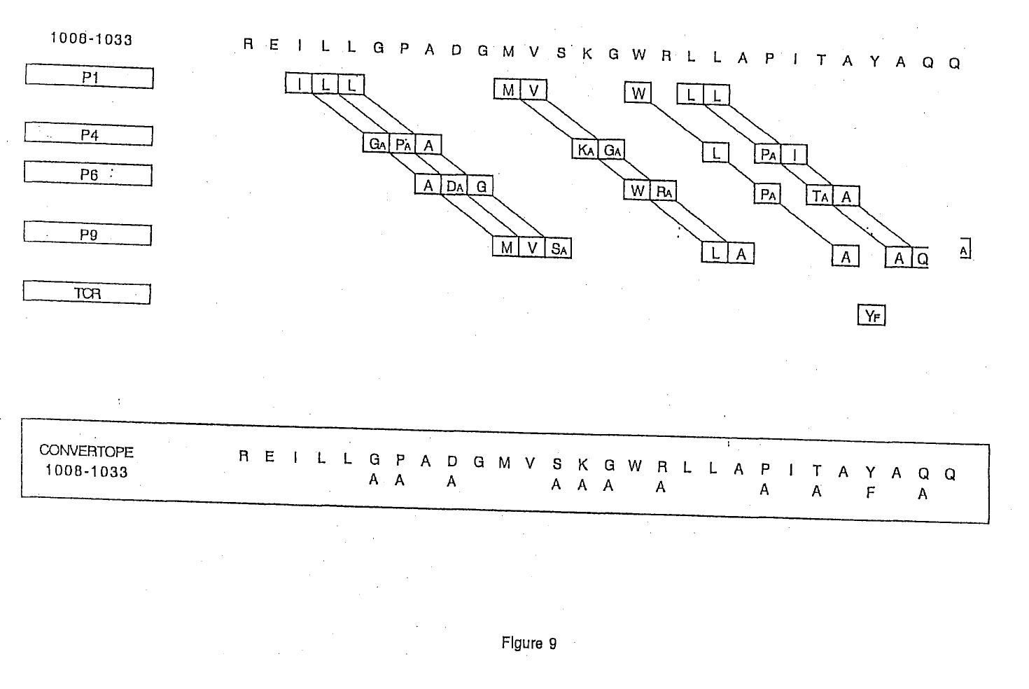

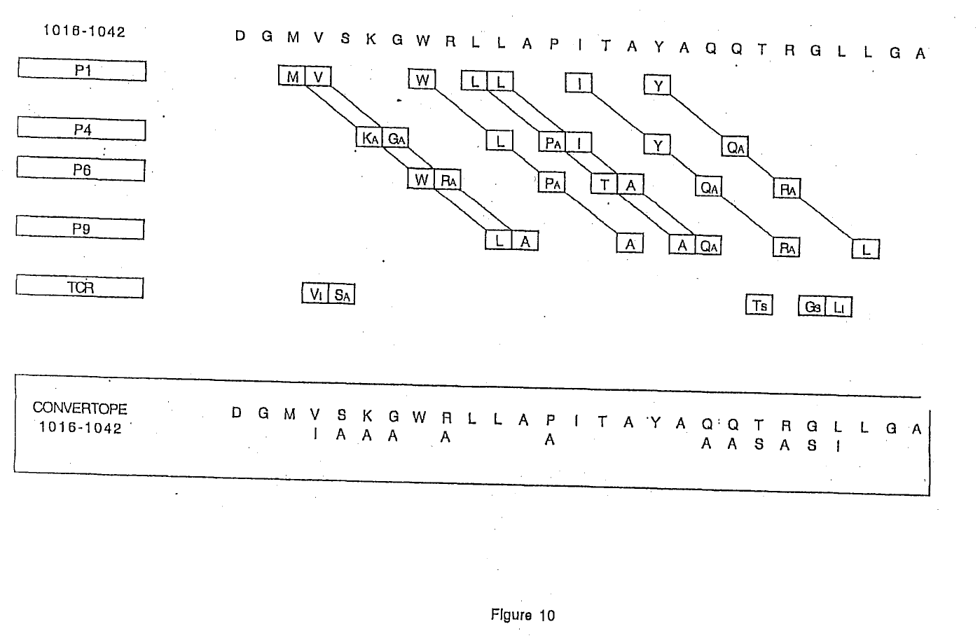

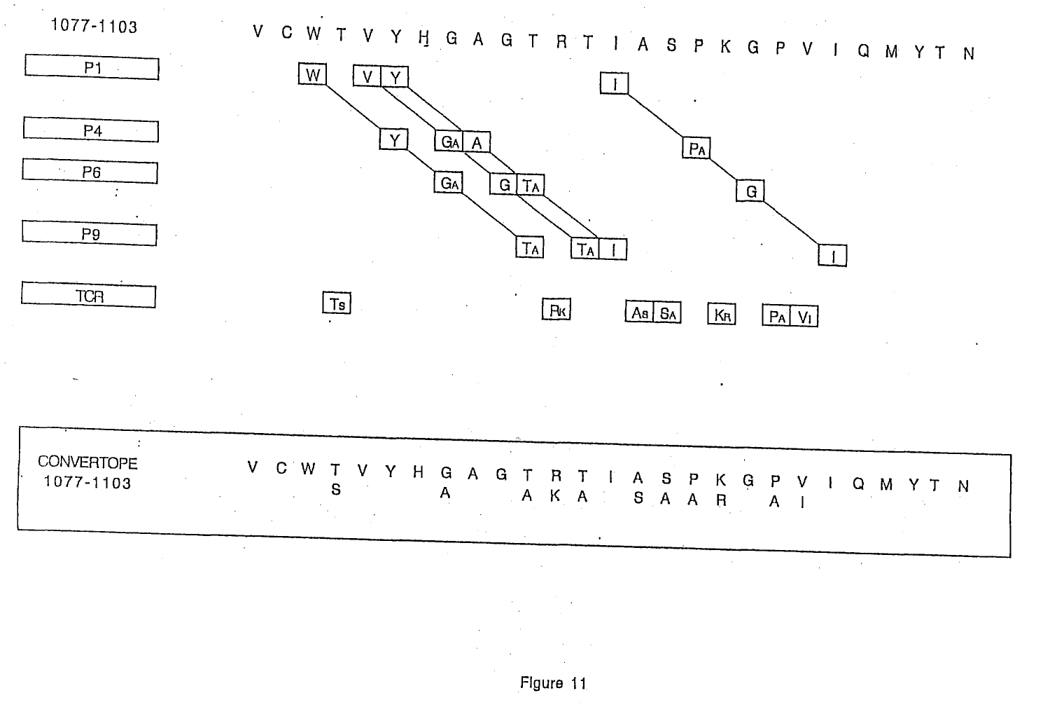

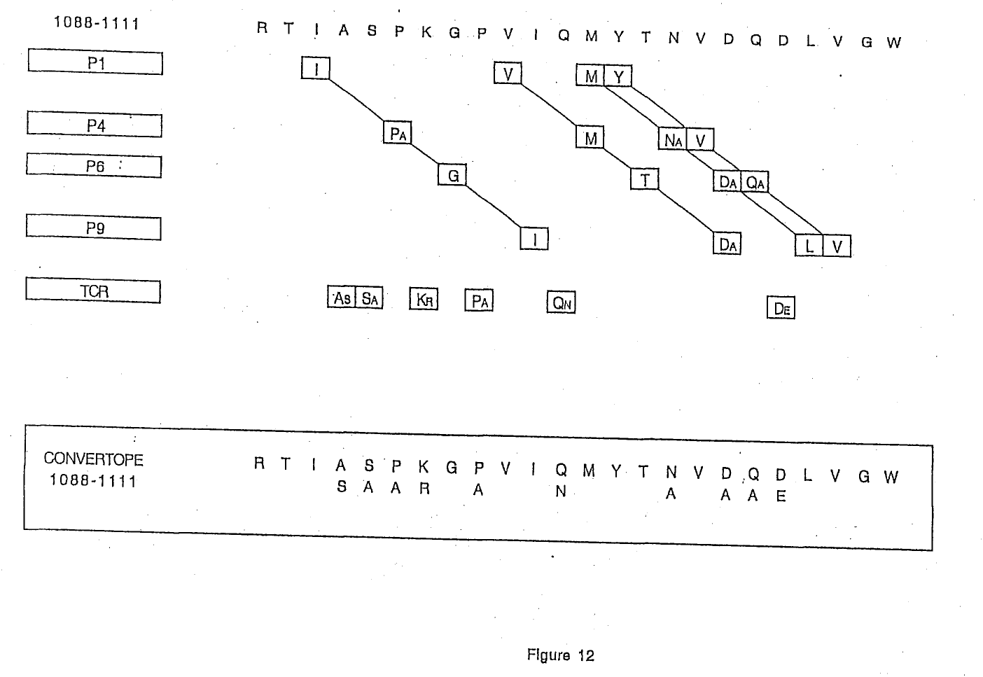

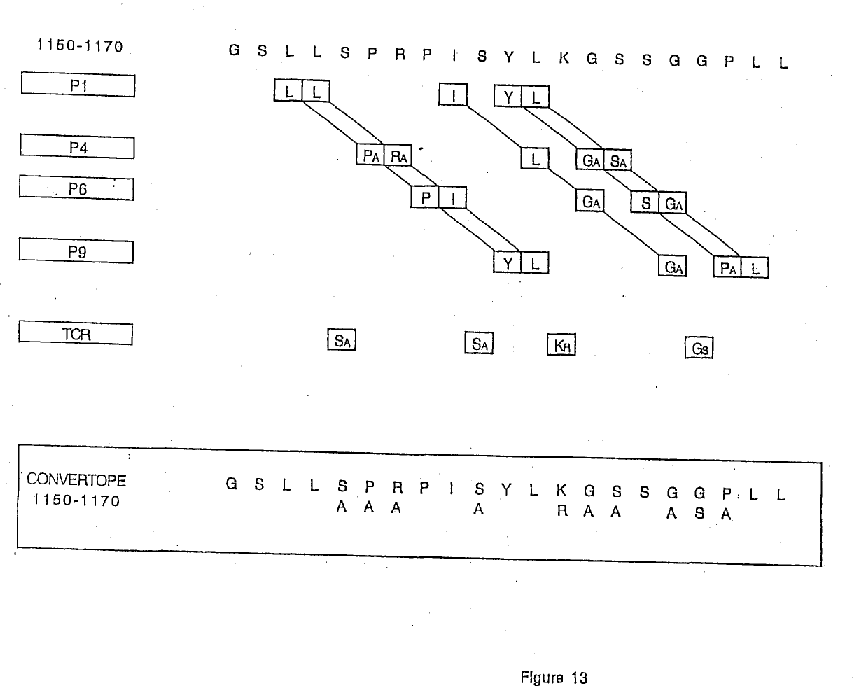

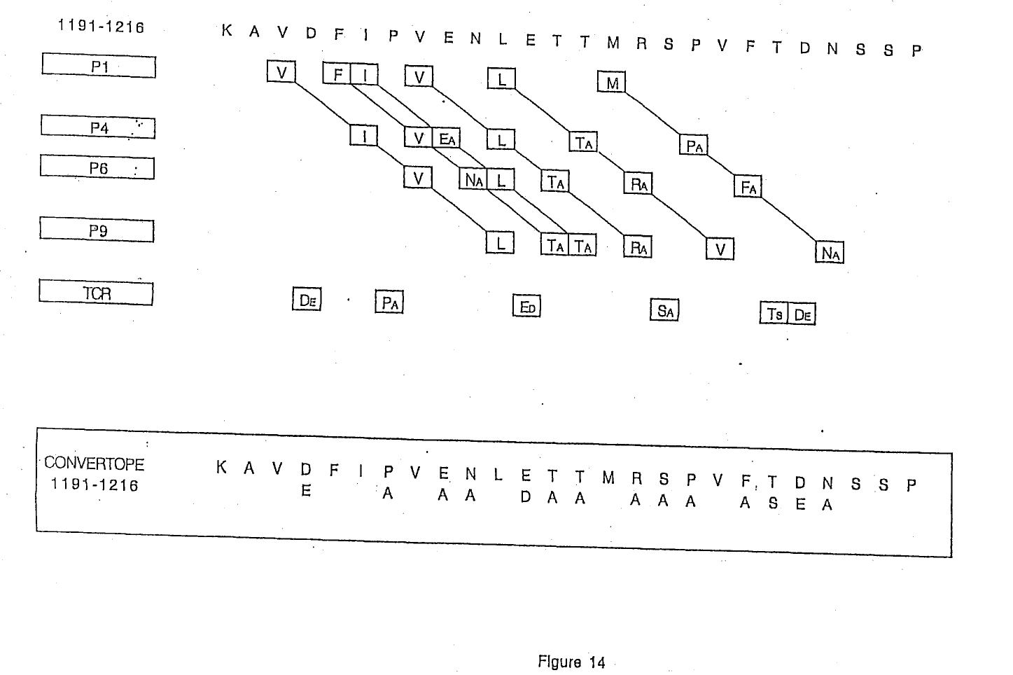

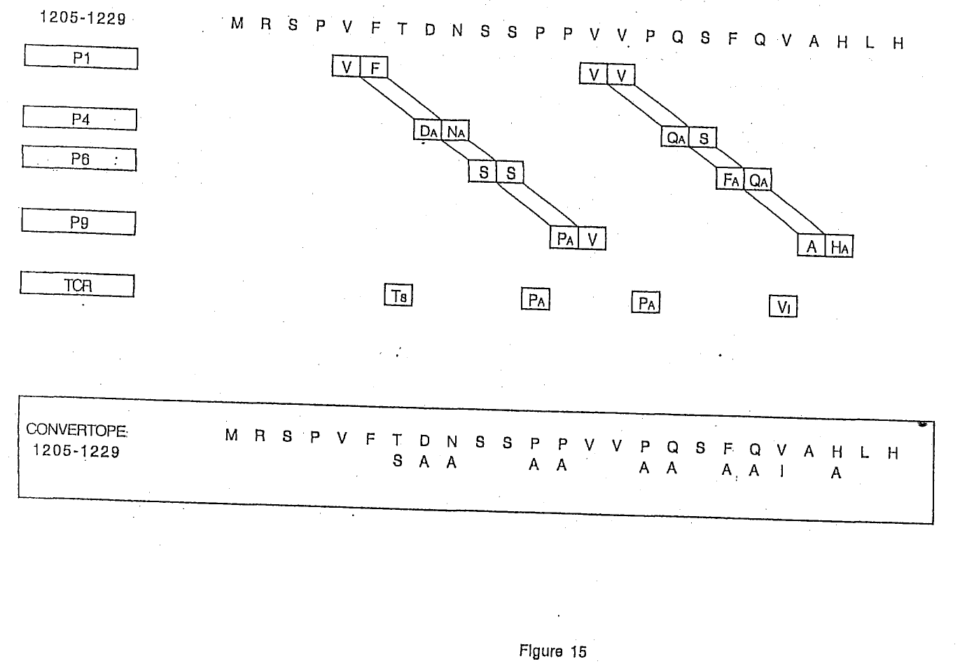

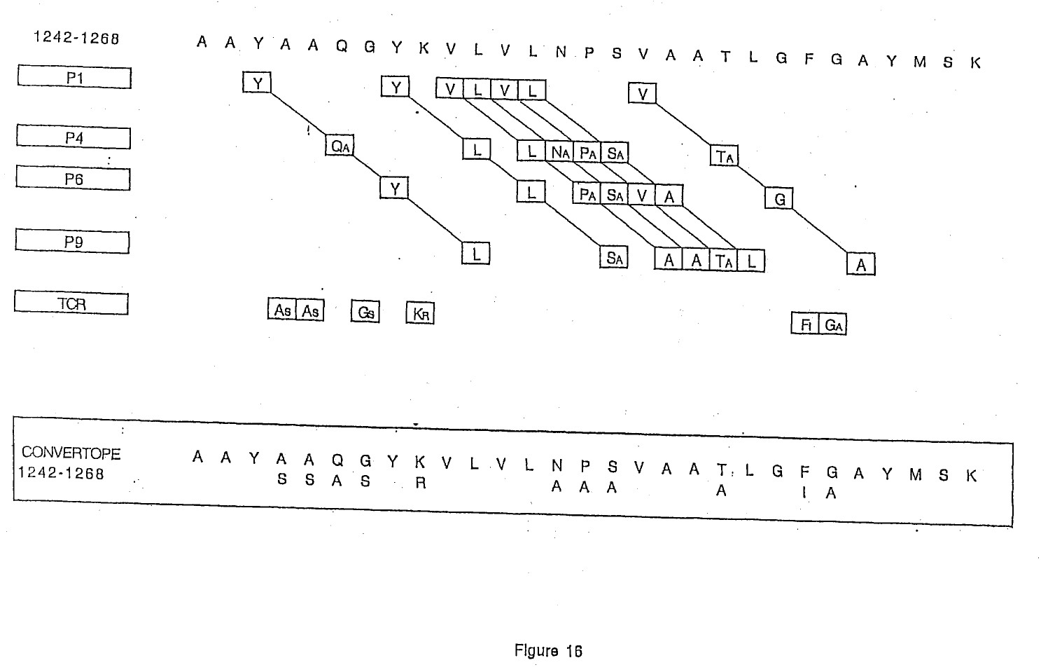

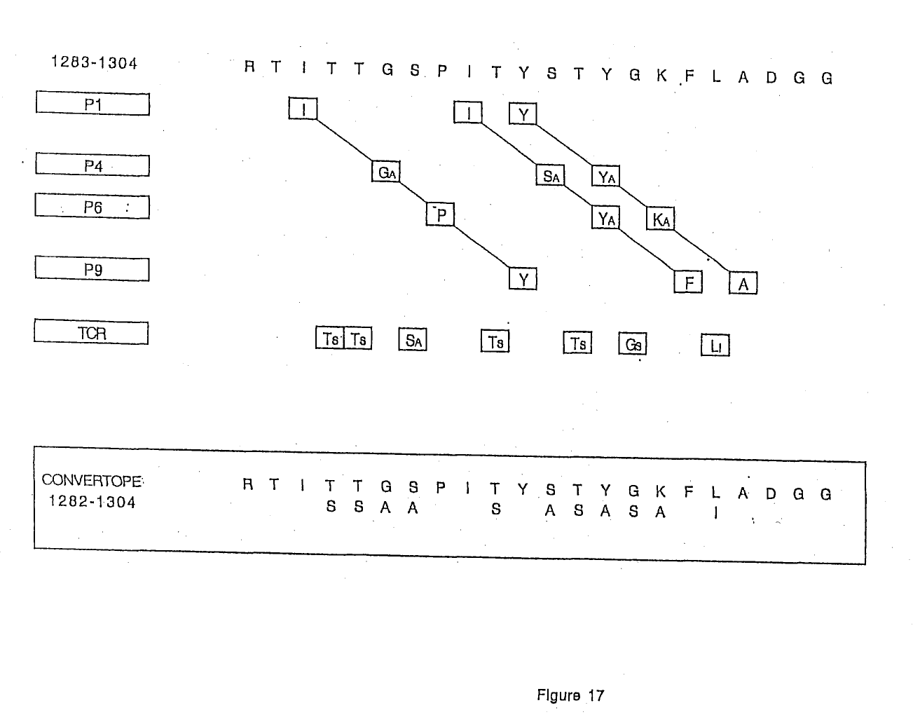

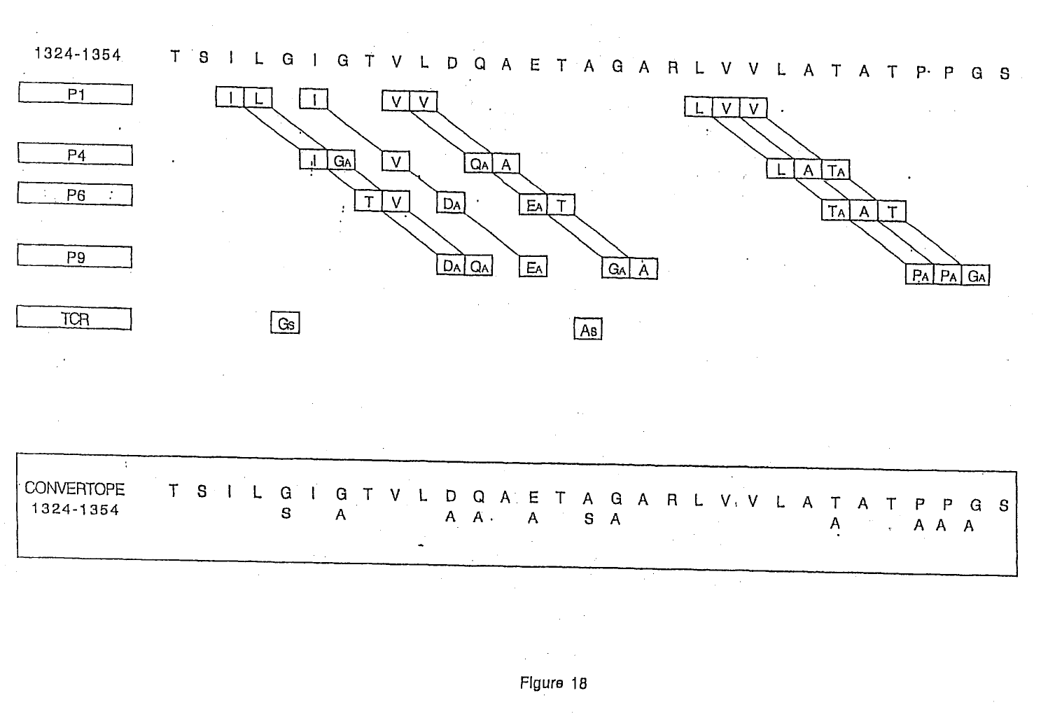

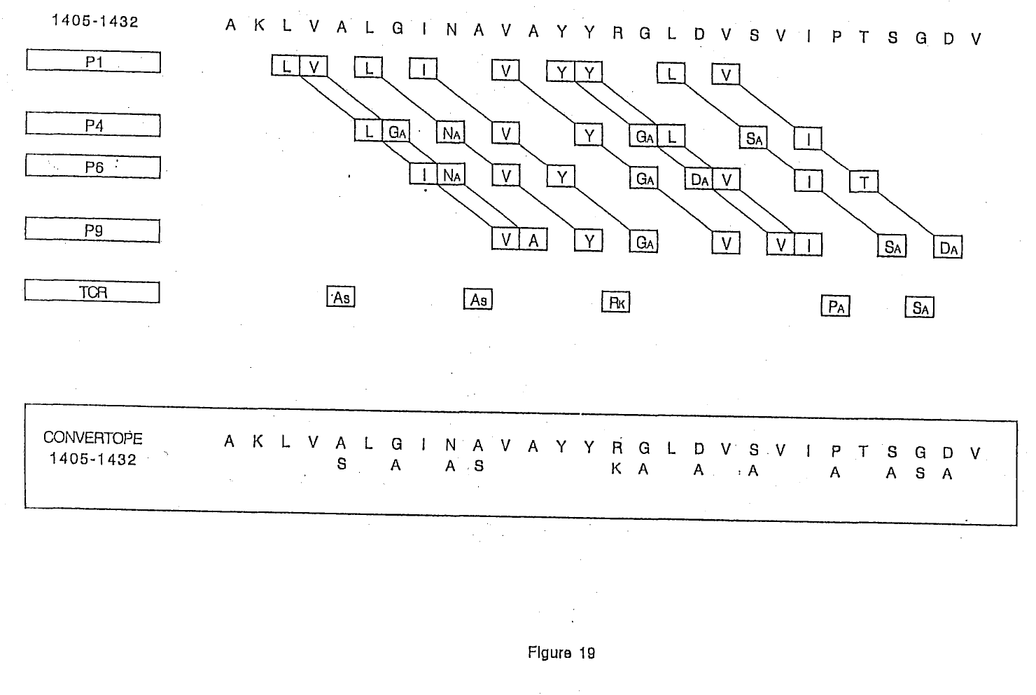

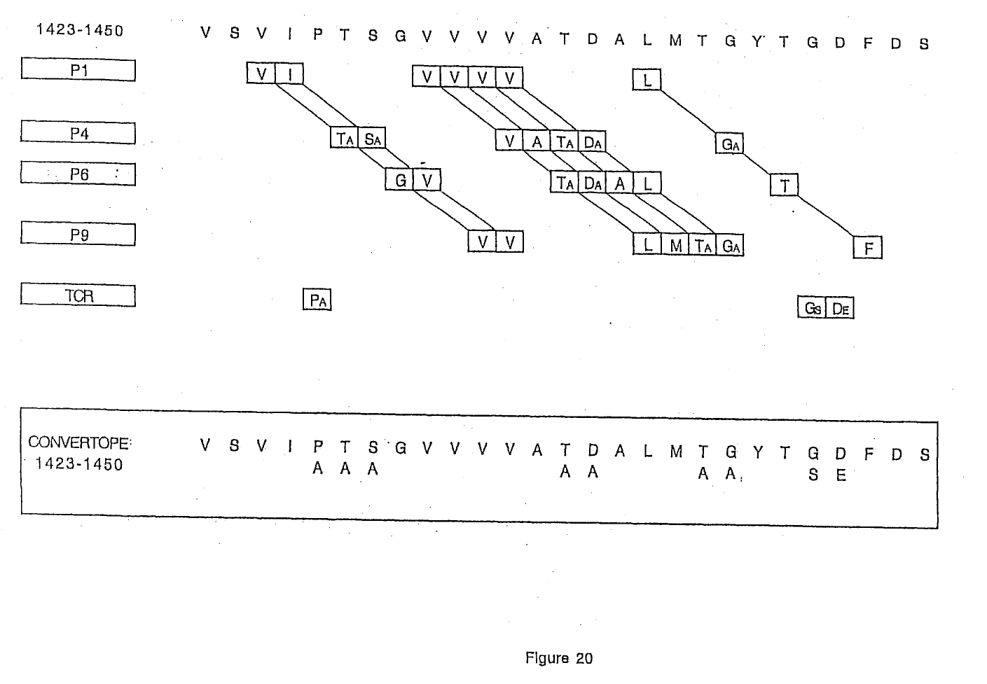

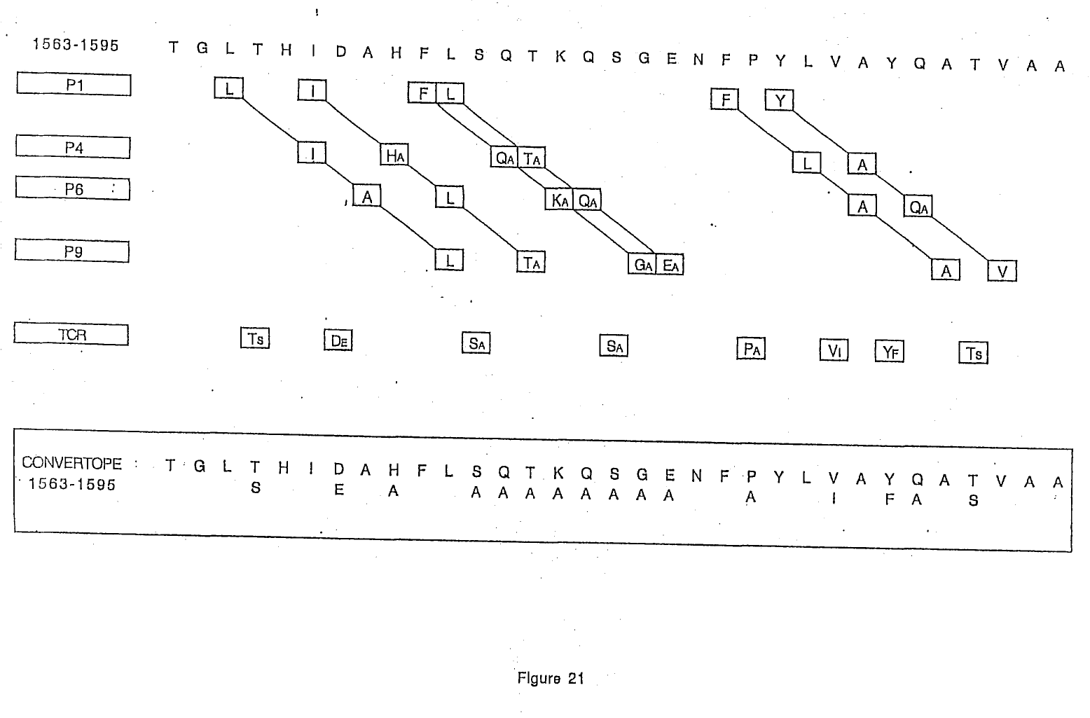

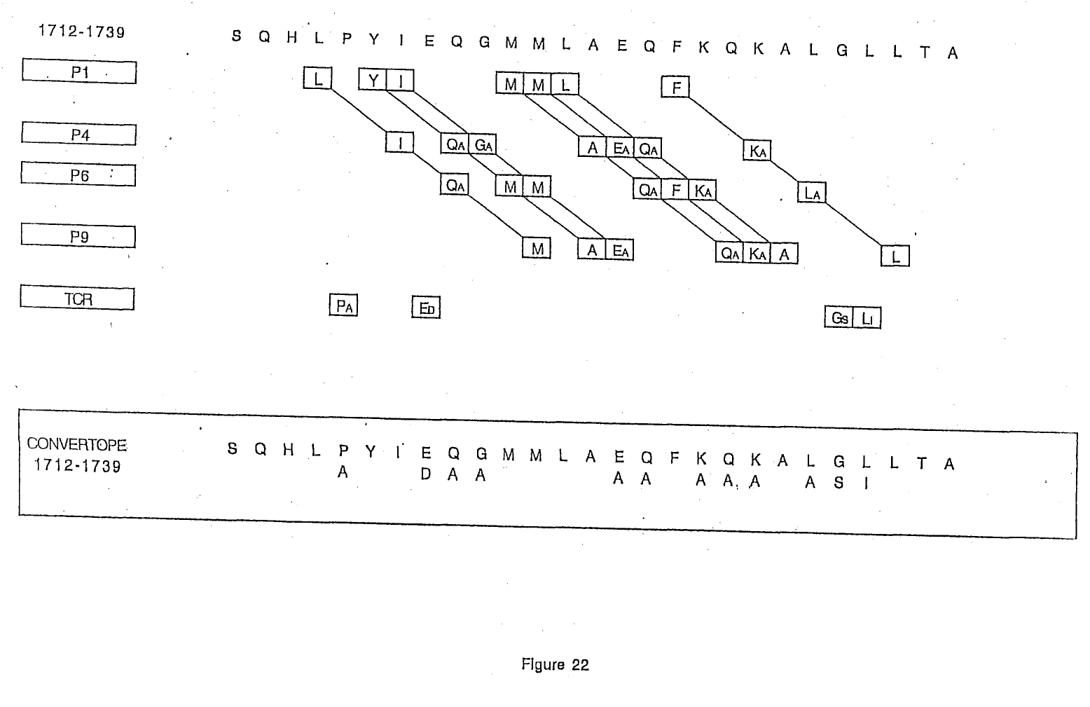

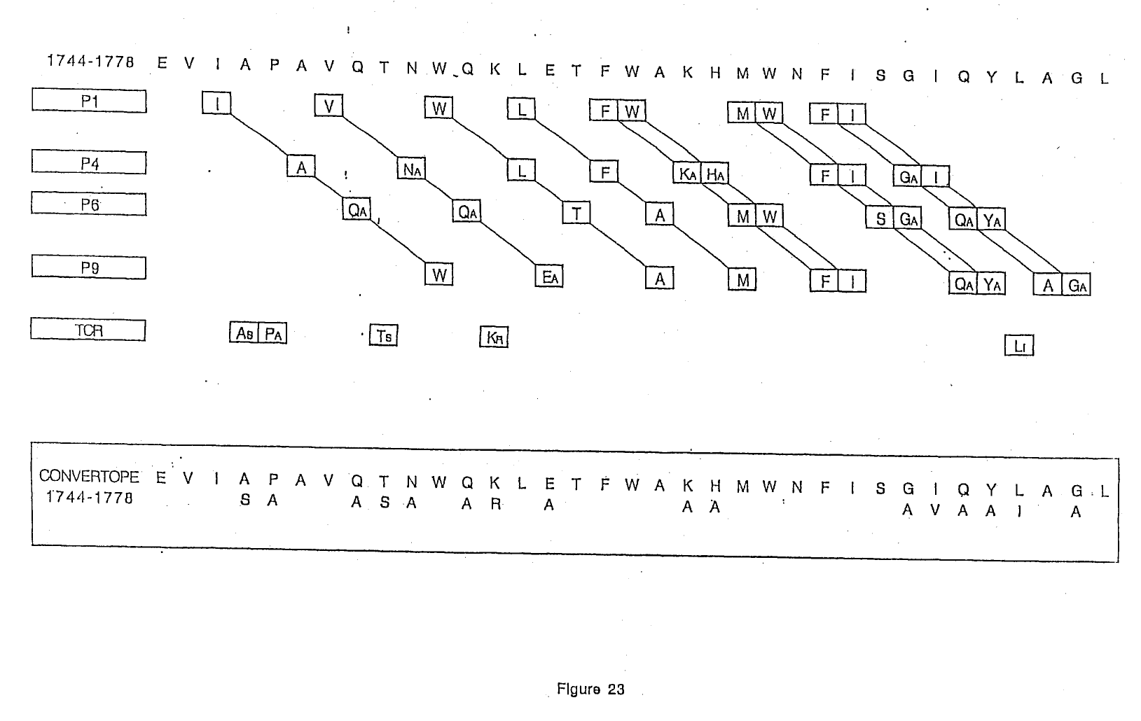

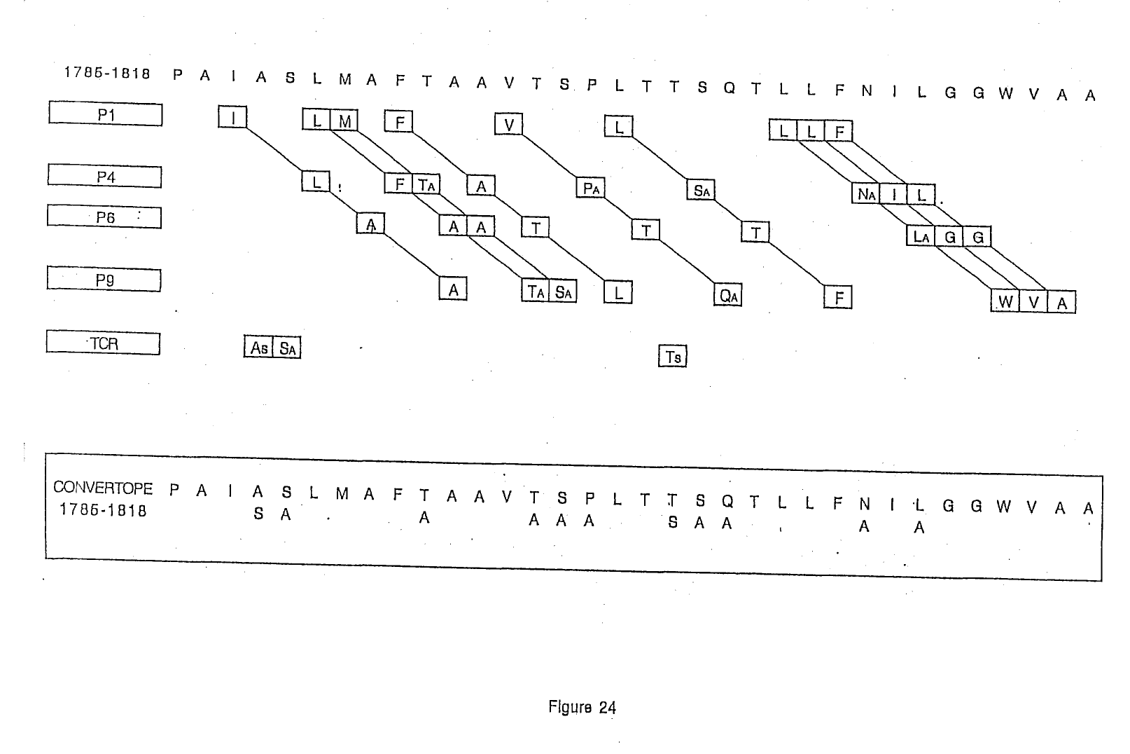

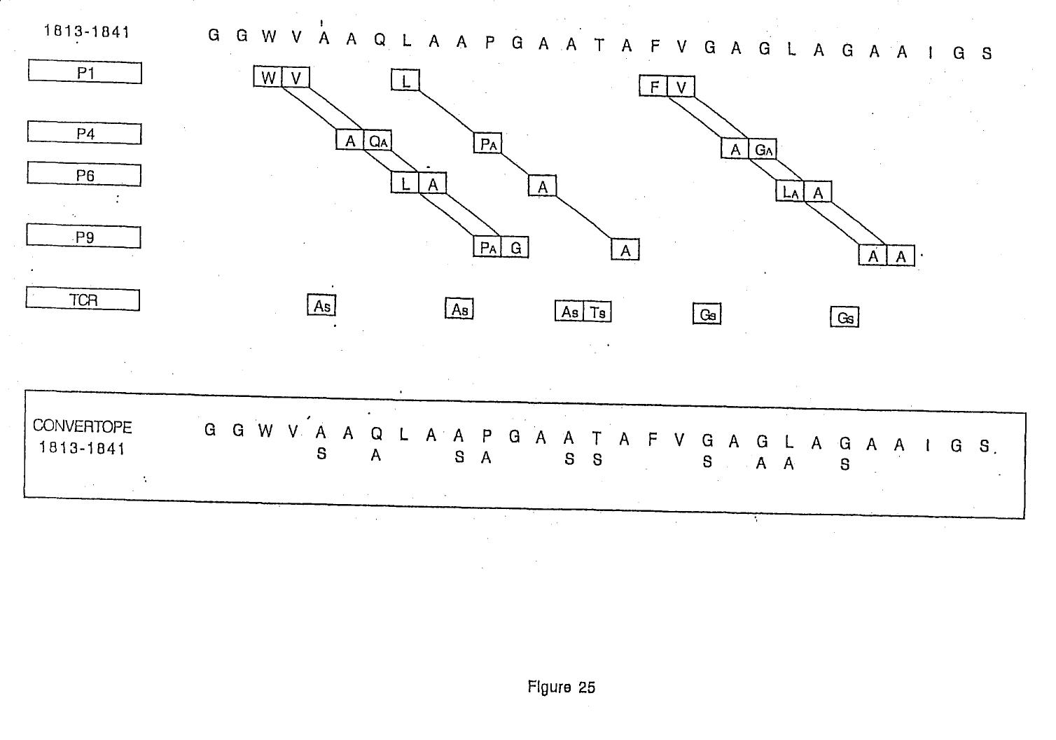

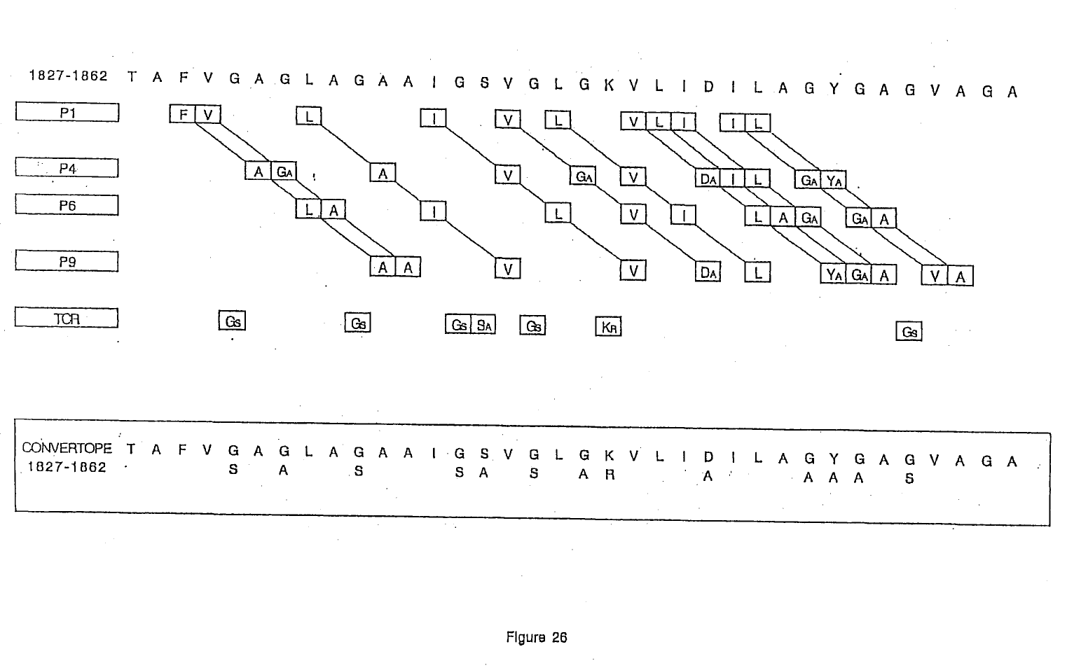

In the 26 sequences (sequences ID Nos. 1 to 26, annexed) selected as potential T epitopes, the substitutions chosen according to the criteria described earlier result in the 26 convertopes presented in Table I hereinafter and sequences ID Nos. 27 to 52, the design of which will be clarified in Example 1 and FIGS. 1 to 26. In sequences ID 27 to 52, the amino acids of a variable nature are designated by Xaa, by convention. It should be pointed out that each convertope includes the peptide with native sequence and all the peptides with totally or partially substituted sequence, at the positions indicated.

TABLE I

CONVERTOPE

| | |
|---|---|
| 20-48 | QDVKFPGGGQIVGGVYLLPRRGPRLGVRA |
| | R ASASA AA AAA AA A |
| 27-53 | GGQIVGGVYLLPRRGPRLGVRATRKTS |
| | SA AAA AA AIAS KRA |
| 35-60 | YLLPRRGPRLGVRATRKTSERSQPRG |
| | AAA K A ASAKAAAA |
| 68-86 | ARRPEGRTWAQPGYPWPLY |
| | SNA SAA A |
| 84-104 | PLYGNEGCGWAGWLLSPRGSR |
| | SAA AS S AAAASAA |
| 114-136 | RRSRNLGKVIDTLTCGFADLMGY |
| | A AA AAA EI |
| 128-149 | AGFADLMGYIPLVGAPLGGAAR |
| | SE A A A AS |
| 154-172 | GVRVLEDGVNYATGNLPGA |
| | K DAA A AAR A |
| 1008-1033 | REILLGPADGMVSKGWRLLAPITAYAQQ |
| | AA A AAA A A A F A |
| 1016-1042 | DGMVSKGWRLLAPITAYAQQTRGLLGA |
| | IAAA A A AASASI |
| 1077-1103 | VCWTVYHGAGTRTIASPKGPVIQMYTN |
| | S A AKA SAAR AI |
| 1088-1111 | RTIASPKGPVIQMYTNVDQDLVGW |
| | SAAR A N A AAE |
| 1150-1170 | GSLLSPRPISYLKGSSGGPLL |
| | AAA A RAA ASA |
| 1191-1216 | KAVDFIPVENLETTMRSPVFTDNSSP |
| | E A AA DAA AAA ASEA |
| 1205-1229 | MRSPVFTDNSSPPVVPQSFQVAHLH |
| | SAA AA AA AAI A |
| 1242-1268 | AAYAAQGYKVLVLNPSVAATLGFGAYMSK |
| | SSAS R AAA A IA |
| 1282-1304 | RTITTGSPITYSTYGKFLADGG |
| | SSAA S ASASA I |
| 1324-1354 | TSILGIGTVLDQAETAGARLVVLATATPPGS |
| | S A AA A SA A AAA |
| 1405-1432 | AKLVALGINAVAYYRGLDVSVIPTSGDV |
| | S A AS KA A A A ASA |
| 1423-1450 | VSVIPTSGVVVVATDALMTGYTGDFDS |
| | AAA AA AA SE |
| 1563-1595 | TGLTHIDAHFLSQTKQSGENFPYLVAYQATVAA |
| | S E A AAAAAAAA A I FA S |
| 1712-1739 | SQHLPYIEQGMMLAEQFKQKALGLLTA |
| | A DAA AA AAA ASI |
| 1744-1778 | EVIAPAVQTNWQKLETFWAKHMWNFISGIQYLAGL |
| | SA ASA AR A AA AVAAI A |
| 1785-1818 | PAIASLMAFTAAVTSPLTTSQTLLFNILGGWVAA |
| | SA A AAA SAA A A |
| 1813-1841 | GGWVAAQLAAPGAATAFVGAGLAGAAIGS |
| | S A SA SS S AA S |
| 1827-1862 | TAFVGAGLAGAAIGSVGLGKVLIDILAGYGAGVAGA |
| | S A S SA S AR A AAA S |

Peptide synthesis is carried out in two stages: on one hand, synthesis of the peptide of the native sequence and, on the other hand, synthesis of the convertope. These syntheses are carried out in a conventional automatic synthesizer.

The group of peptides constituting a convertope is obtained in a single synthesis during which, for each cycle corresponding to a substitutable position, use is made of a mixture of the native amino acid and of the substitution amino acid, the concentrations of which are adjusted to obtain an equimolar ratio in the synthesized peptide.

The process according to the invention was developed for the hepatitis C virus and, more precisely, for the capsid protein and the NS3 and NS4 proteins of this virus.

It is quite clear that the same process for preparing immunogenic peptides including restricted regions of chosen viral proteins and convergent combinatory peptide libraries is applicable to any virus involving a T cell immune response.

Thus, synthetically, this process includes:

a) identifying sequences of amino acids constituting potential T epitopes, by predictive, non-experimental searching—for anchoring motifs for class II MHC molecules, from the native sequences of the proteins of the virus—and for areas having a high density of said anchoring motifs;

b) designing convergent combinatory peptide libraries (termed “convertopes”), derived from the sequences of epitopes identified in a), by targeted substitution of amino acids at chosen positions, to induce non-natural degeneracy of each of the epitopes,

on one hand, in order to increase the ability of the epitope to anchor to the class II MHC molecules,

and, on the other hand, in order to broaden the repertory of recognition of the epitope by the T cell receptor;

c) synthesizing

on one hand, the peptides with native sequence chosen among the peptides as identified in a),

and, on the other hand, the corresponding convertopes as identified in b).

The search for anchoring motifs is carried out by applying the following criteria which are common to the majority of class II MHC alleles:

in a motif of 9 adjacent amino acids, the amino acid in position P1 is necessarily hydrophobic and is an aliphatic or aromatic amino acid, i.e. V, I, L, F, M, W or Y;

the amino acids in positions P4, P6 and P9 are, preferably, I, V, L, M, F or A; A, P, G, S or T; and A, I, V, Y, L, F or M, respectively.

Designing convertopes includes substituting, in each identified anchoring motif, one or more of the P4, P6 or P9 residues if they fail to satisfy the common criteria as defined earlier, with a more compliant amino acid and, preferably, with an alanine.

Designing convertopes can further include substituting one or more residues other than 1, 4, 6 or 9, according to a matrix of replaceability favoring recognition of the epitope by the T cell receptor, except for residues common to 2 or more neighboring anchoring units the substitution of which would be incompatible with the adjacent unit or with a substitution already effected therein.

The mixtures of imunogenic peptides obtained with the process according to the invention are chiefly intended for vaccination against the viruses from which these peptides derive.

More precisely, the present invention relates to a composition designed for vaccination against the hepatitis C virus. This composition includes pairs of native sequences and of degenerated sequences obtained using the process according to the invention and chosen from among the convertopes presented in Table I.

The vaccinal composition can find applications that are not only prophylactic but also therapeutic to stimulate the immune reactions of patients already infected.

The following examples serve to illustrate the invention without, however, limiting the scope thereof.

### EXAMPLE 1

Design of the Convertopes

Bibliographic data have led to the choice, as proteins potentially of interest in preparing immunogenic peptides constituting potential T epitopes, of the capsid protein and the NS3 and NS4 proteins of the hepatitis C virus.

The sequences of these proteins are available from the SWISS-PROT data bank.

The process for identifying potential T epitopes and for designing, on the base of these, convergent peptide libraries or “convertopes” includes the following steps:

First Step.

Potential anchoring motifs are identified on the basis of the rules common to the majority of the alleles of HLA-DR human class II MHC molecules, that is to say an aliphatic or aromatic amino acid (V, I, L, F, M, W, Y) in the first position of a portion of 9 residues, capable of interacting with pocket P1. As regards the HLA-DR alleles, this pocket P1 is only slightly polymorphous (presence of a single Val/Gly dimorphism in position 86 in the P chain) and represents a rule of recognition shared by all the alleles. In addition, the presence of a hydrophobic aliphatic or aromatic residue in position P1 appears to be an essential condition for interaction with the corresponding anchoring pocket, with this anchoring playing a dominant role in the interaction of the antigenic peptide with the MHC molecule by permitting, in particular, the formation of a stable complex.

Identification of the amino acid residues potentially involved in anchoring in the area of anchoring pockets P4, P6, P9 derives mathematically from the first position. For each anchoring position, a consensus has been defined, on the basis of the analysis of the results of STURNIOLO T. (Nature Biotech., 1999, 17:555), by looking for major amino acids capable of interacting with the greatest number of different alleles. The following table sets out the specific features of the four pockets P1, P4, P6 and P9 of the HLA-DR class II molecules of the MHC.

TABLE II
Consensus anchoring motif for class II MHC molecules

| P1 | P4 | P6 | P9 |
|---|---|---|---|
| YFMIVLWM | IVLMFA | APGST | AIVYLFM |

The portions of sequences of the capsid and NS3 and NS4 proteins having a high density of these anchoring motifs were identified and selected. They include 1 or 2 amino acids upstream of the first anchoring unit and downstream of the last one. These ends can be extended by 1 to 6 additional natural amino acids in order to include, in the sequence, charged amino acids capable of facilitating the solubility of sequences that are too rich in hydrophobic residues.

In all, 26 sequences were thus chosen: in the capsid protein, the sequences ID Nos. 1 to 8; in the NS3 protein, the sequences ID Nos. 9 to 21; in the NS4 protein, the sequences ID Nos. 22 to 26.

Second Step.

The potential anchoring motifs obtained were compared with the consensus anchoring motif for the class II MHC defined earlier.

If the native amino acid does not meet the conditions set forth in Table II, an alanine is systematically introduced into positions P4, P6, P9. An alanine is, indeed, capable of replacing the amino acids involved in anchoring to the class II MHC molecules (Fleckenstein B. et al, Eur. J. Bioch. 1996. 240:71, Sturniolo et al., Nature Biotech 1999, 17:555), by making it possible to remove the negative influence of a side chain and thus to improve the association with the MHC molecules without affecting recognition by the T cells (Ahlers et al., Proc. Natl. Acad. Sci. USA. 1997. 94:10856). However, if the native amino acid is involved in an anchoring position P1 of an overlapping, neighboring anchoring motif, substitution is not contemplated.

Third Step.

In the case of the amino acids not involved in potential anchoring to the class II MHC molecules and liable to be involved in the interaction with the specific receptor of the T cells, that is to say positions P2, P3, P5, P7 and P8, substitution of one of these positions can be effected, said substitution being dedicated to specific recognition by the T cells. The purpose of these substitutions is to generate variants related to the native peptides capable of generating a broader number of T cells specific of the native antigen. This broadering is contemplated in view of the considerable degeneracy of T recognition (Hemmer B., Immunol. Today 1998 19:163). A clonal T cell has, indeed, the ability to recognize a very large number of peptide antigens that are related, but also not related, to the native sequence with, in certain cases, better affinity.

The substitutions carried out are based on a matrix of replaceability of the Geysen type (Geysen M. et al., J. Mol. Recog. 1998. 1, 32-41). This matrix of replaceability was prepared on the basis of peptide/antibody recognition, which is related to the mode of recognition of the receptor of the T cells. The amino acids in positions P2, P3, P5, P7 and P8 can be substituted unless they are involved in an anchoring motif located in a neighboring unit. Given the various replacement possibilities suggested by Geysen's matrix, substitutions will preferentially concern the amino acid with one of the best replaceability indexes.

The design of the convertopes is schematically represented in FIGS. 1 to 26.

Legend of FIGS. 1 to 26

The native sequence of interest is presented in the upper part of the figure.

The amino acids involved in the potential reaction with an anchoring pocket are represented by a

[symbol]

The MHC anchoring points P1, P4, P6 and P9 are associated within an anchoring motif in a diagonal mode of representation.

The substitutions of natural amino acids, according to the rational method explained in the description, are indicated in subscript form.

The amino acids substituted to improve recognition by the T cell receptor (TCR) are also shown.

The addition of the two substitution approaches (MHC and TCR) is recapitulated in the lower, framed part of the figure.

### EXAMPLE 2

A) Preparation of the Peptides

The peptides are synthesized using the conventional solid phase strategy of the Boc-benzyl (or Fmoc) type, in an automated peptide synthesizer (model 430A, APPLIED BIOSYSTEM, INC.). The side chain protection groups are as follows: Asn(Trt), Gln(Trt), Asp(Ochx), Glu(Ochx), Ser(Bzl), Thr(Bzl), Cys(4-MeBzl) and His(Dnp) (SHEPPARD R. C. et al., Peptide Synthesis Comp. Org. Chem., 1979, 5:321). The amino acids are introduced using the HBTU/HOBt activation protocol with double systematic coupling on a Boc-X-Pam resin (where X represents the amino acid in C-terminal position). After thiolysis of the dinotrophenyl group Dnp, followed by final deprotection and cleaving with hydrofluoric acid, the cleaved, deprotected peptide is precipitated with cold diethyl ether, and then dissolved in 5% acetic acid and freeze dried. The peptide is purified to over 90% on a preparative column of 5 mm×250 mm, 100a Nucleosyl C18, RP-HPLC (MACHERY NAGEL, Duren, Germany). Homogeneity is confirmed by analytical HPLC on a Vydac column eluted with a system of solvents (trifluoroacetic acid-acetonitrile-water). The identity of the peptide is confirmed by determining the amino acid composition and by mass spectrometry recorded on a Bio Ion 20 plasma mass spectrometer (BIO ION AB, Uppsala, Sweden).

B) Preparation of the Convertopes.

Synthesis is carried out according to the method described previously except for the degenerated positions, where equimolar quantities of protected amino acids are used in the coupling reactions instead of a single amino acid, as in a conventional synthesis. To compensate for the kinetic differences in the reactivity levels of the different amino acids, a first coupling is carried out with 1 mmol (total quantity) of Boc-amino-acid (or of a mixture). A second coupling using 2 mmol (total quantity) is then systematically carried out. After cleaving, the raw peptide is dissolved in trifluoroacetic acid and precipitated in a solution of cold diethyl ether. After centrifugation, the precipitate is dissolved in water and then purified by gel filtration on a TSK HW40S column (MERK, Darmstadt, Germany). An aliquot is subjected to acid hydrolysis in order to determine the amino acid composition.

### EXAMPLE 3

Evaluation of the Immunogenicity of the Mixtures

The sequences of the native peptides and of the convertopes deriving therefrom are presented in the listing of the sequences and in Table II. These products, and different combinations thereof, will be used as antigens for immunization purposes.

The immunogenicity of the peptides and the convertopes is evaluated taking two complementary approaches: on one hand, by immunization in vivo of humanized mice; on the other hand by immunization in vitro of human cells from HLA-typed donor human.

A) Mice deficient of murine class II MHC molecules (APO) and transgenic in respect of various human class II MHC molecules (DR1, DR2, DR3, DQ6, DQ8) are immunized with the mixtures of peptides chosen, using three mice per condition. The mice are immunized with 100 micrograms of peptides in a volume of 100 microliters of a mixture of water/Freund complete adjuvant administered subcutaneously. The mice are restimulated one or two times at 15 day intervals with 50 micrograms of peptides in 100 microliters of a mixture of water/Freund incomplete adjuvant. A serum sample of the immunized mice is recovered prior to each injection in order to evaluate the production of specific antibodies, as well as the production of cytokines in vivo. One week after the last injection, the ganglions, as well as the spleens, of the different groups of animals are collected, pooled on a group basis and then cultivated in order to study the ability to induce cell proliferation in vitro and the ability to induce the production of TH1 or TH2 type cytokines in vitro, so as to identify the peptides and the convertopes having the best immunogenicity.

B) Immunization in vitro was carried out as follows: CD14+ blood monocytes isolated by positive selection are differentiated into dendritic cells after maintaining in contact with IL4 and GM-CSF for 5 days. The CD4+ cells isolated from the same donor are then immunized in vitro with the different mixtures of peptides and convertopes. One subsequent restimulation is carried out under the same conditions, and then the B lymphocytes of the same donor are used as antigen-presenting cells. The production of different cytokines is sought for in the culture supernatants, 24 and 48 hours after immunization in vitro.

1. Process for preparing immunogenic peptides corresponding to restricted regions of virus proteins involving a T cell immune response and to convergent combinatory peptide libraries derived from, said regions, characterized in that it includes:
a) identifying sequences of amino acids constituting potential T epitopes, by predictive, non-experimental searching—for anchoring motifs for class II MHC molecules, from the native sequences of the proteins of the virus—and for areas having a high density of said anchoring motifs;
b) designing convergent combinatory peptide libraries (termed “convertopes”), derived from the sequences of epitopes identified in a), by targeted substitution of amino acids at chosen positions, to induce non-natural degeneracy of each of the epitopes,
on one hand, in order to increase the ability of the epitope to anchor to the class II MHC molecules,
and, on the other hand, in order to broaden the repertory of recognition of the epitope by the T cell receptor;
c) synthesizing
on one hand, the peptides with native sequence chosen among the peptides as identified in a),
and, on the other hand, the corresponding convertopes as identified in b).

2. Process according to claim 1, characterized in that the search for anchoring motifs is carried out by applying the following criteria which are common to the majority of class II MHC alleles:
in a motif of 9 adjacent amino acids, the amino acid in position P1 is necessarily hydrophobic and is an aliphatic or aromatic amino acid, i.e. V, I, L, F, M, W or Y;
the amino acids in positions P4, P6 and P9 are, preferably, I, V, L, M, F or A; A, P, G, S or T; and A, I, V, Y, L, F or M, respectively.

3. Process according to claims 1 or 2, characterized in that designing convertopes includes substituting, in each identified anchoring motif, one or more of the P4, P6 or P9 residues if they fail to satisfy the common criteria as defined in claim 2, with a more compliant amino acid and, preferably, with an alanine.

4. Process according to claim 1 or 3, characterized in that it further includes substituting one or more residues other than 1, 4, 6 or 9, according to a matrix of replaceability favoring recognition of the epitope by the T cell receptor, except for residues common to 2 or more neighboring anchoring units the substitution of which would be incompatible with the adjacent unit or with a substitution already effected therein.

5. Process according to any one of claims 1 to 4, characterized in that each convertope is obtained in a single synthesis, during which, for each cycle corresponding to a substitutable position, use is made of a mixture of the native amino acid and of the substitution amino acid, adjusted to obtain an equimolar ratio in the synthesized peptide.

6. Process according to any one of claims 1 to 5, characterized in that the proteins of the virus are the capsid protein and the NS3 and NS4 proteins of the hepatitis C virus (HCV).

7. Group of potential epitope sequences identified using the process according to claim la), including sequences ID Nos. 1 to 8 of the capsid protein, sequences ID Nos. 9 to 21 of the NS3 protein and sequences ID Nos. 22 to 26 of the NS4 protein of the HCV.

8. Convertopes derived from the sequences according to claim 7 using the process according to claims 2 and 3, as represented in sequences ID Nos. 27 to 52.

9. Composition based on a mixture of immunogenic peptides intended for vaccination against the hepatitis C virus, characterized in that it includes pairs of native sequences and of degenerated sequences (convertopes) obtained using the process according to any one of claims 1 to 6, chosen from among the sequences according to claim 7 and the convertopes according to claim 8.
